(19) United States
(12) Patent Application Publication
(10) Pub. No.: US 2018/0051257 A1
(43) Pub. Date: Feb. 22, 2018

(54) METHODS FOR GENERATING ALVEOLAR TYPE II CELLS

(71) Applicants: Sirous Sadeghian Chaleshtori, Tehran (IR); Mohammad Reza Mokhber Dezfouli, Tehran (IR); Hossein Baharvand, Tehran (IR); Yaser Tahamtani, Tehran (IR)

(72) Inventors: Sirous Sadeghian Chaleshtori, Tehran (IR); Mohammad Reza Mokhber Dezfouli, Tehran (IR); Hossein Baharvand, Tehran (IR); Yaser Tahamtani, Tehran (IR)

(21) Appl. No.: 15/653,489

(22) Filed: Jul. 18, 2017

(30) Foreign Application Priority Data
Aug. 20, 2016 (IR) 13955014000300646

(51) Int. Cl. C12N 5/071

(52) U.S. Cl. CPC C12N 5/0688; C12N 2501/999; C12N 2533/54; C12N 2506/02; C12N 2500/84; C12N 2501/115

(57) ABSTRACT

A method for generating alveolar type II (AT II) cells or lung epithelial cells is disclosed. The method comprises a two-step differentiation protocol to generate alveolar type II (AT II) cells from one or more embryonic stem cells (mESC). The two-step differential protocol is performed by first step of, inducing the embryonic stem cells (mESC) by a definitive endoderm (DE) inducer to produce mESC-DE cells at a first period of time. The second step includes, inducing the mESC-DE cells by one or more growth promoting factors at a second period of time to generate AT (II) cells. The method for generating AT (II) cells from the embryonic stem cells (mESC) ensures abundant production of AT (II) cells for the treatment of pulmonary diseases. Moreover, the main function of AT (II) cells is the production of surfactants, which could be used for the treatment of lungs related diseases.

### BACKGROUND OF THE INVENTION

Alveolar cells are cells lining the alveoli, or the hollow cavity of the lungs. There are two types of alveolar cells, Type I alveolar cells, and Type II alveolar cells. The function of Type II alveolar cells is of major importance, secreting a pulmonary surfactant, which decreases the surface tension within the alveoli. These cells are further capable of cellular division, which produces more Type I alveolar cells when the lung tissue is damaged.

Due to their delicate structure, the function of alveolar cells could be affected by various pathological conditions such as, chronic obstructive pulmonary disease (COPD), idiopathic pulmonary fibrosis (IPF), and respiratory distress syndrome (RDS). Rapid and timely diagnosis of the primary symptoms is critical for the patient. However, activities, such as any exercise, performed by an individual will again effect the function of alveolar cells, and the symptoms may reappear in the long term. Whole lung transplantation with 50% success rate for five-year remains the only therapeutic option for most end stage patients affected by such diseases.

Recent studies have shown that directing pluripotent stem cells (PSCs) through a stepwise process can lead to the production of lung epithelium in vitro. However, identifying efficient and defined inductive materials to promote differentiation of epithelial cells of the lung type II (AT II) is an important issue. A number of approaches for differentiation protocols has been developed, each with their own deficiencies.

Mimicking lung developmental stages is an approach for producing ATII cells from pluripotent cell sources in vitro. The first developmental step toward the lung fate includes the formation of anterior de native endoderm (DE). Then, anterior DE cells form lung progenitors, and finally differentiate into lung epithelial cells. Some studies have shown that directing PSCs through a process can lead to the production of lung epithelium in vitro. Several protocols have been developed for DE induction of PSCs and proved the importance of transforming growth factor beta (TGF-β) and Ent3a signaling pathways for DE differentiation of PSCs. Further, PSC derived DE (PSC-DE) cells must be competent to further differentiate into endodermal derived cell types such as hepatocytes, pancreatic and lung cells. However, the generated PSC-DE cells lacks consistency, and exhibit different efficiencies when differentiated into these endodermal cell types.

Thus, there is a clear and present need for a method of generating lungs type II epithelial cells using a natural growth promoting molecule, where it must be compatible with the human body as well. Further, there is a need for a method implemented with simple and efficient steps of inducing the differentiation of alveolar type AT (II) cells and the preterm manifestation of pulmonary surfactant.

### SUMMARY OF THE INVENTION

The present invention relates to a method for generating alveolar type II (AT II) cells or lung epithelial cells, is disclosed. The method comprises a two-step differentiation protocol to generate alveolar type II (AT II) cells from one or more embryonic stem cells (mESC). In an embodiment, the two-step differential protocol is performed by: step A. Inducing the embryonic stem cells (mESC) by a definitive endoderm (DE) inducer to produce mESC-DE cells at a first period of time, and step B. Inducing the mESC-DE cells by at least three growth promoting factors at a second period of time to generate alveolar type II (AT II) cells.

In one embodiment, the definitive endoderm (DE) inducer is an IDE2 inducible molecule. In some embodiments, the growth promoting factors are hydrocortisone, fibroblast growth factor (FGF2), and conditioned medium of A549 cell line. In another embodiment, the growth promoting factor is hydrocortisone alone. In some embodiments, the method further comprises providing embryonic stem cells (mESC) by culturing on one or more gelatin coated dishes. In some embodiments, the first period of time is 6 days, and the second period of time is 9 days. In various embodiments, the method further comprises inducing the mESC-DE cells by said growth promoting factors including hydrocortisone, fibroblast growth factor (FGF2), and conditioned medium of A549 cell line produces 37% of surfactant protein-C (SP-C) expressing cells at step B.

The present disclosure is directed to a method for generating alveolar type II (AT II) cells, comprising: (a) a two-step (step A and B) differentiation protocol to generate alveolar type II (AT II) cells from one or more embryonic stem cells (mESC), wherein the two-step (step A and B) differential protocol is performed by: (i) A. inducing the embryonic stem cells (mESC) by a definitive endoderm (DE) inducer to produce mESC-DE cells at a first period of time, and (ii) B. inducing the mESC-DE cells by at least three growth promoting factors at a second period of time to generate alveolar type II (AT II) cells.

In one embodiment, the definitive endoderm (DE) inducer is an IDE2 small inducible molecule. In another embodiment, the growth promoting factors are hydrocortisone, fibroblast growth factor (FGF2), and conditioned medium of A549 cell line. In one embodiment, the method further comprises providing embryonic stem cells (mESC) by culturing on one or more gelatin coated dishes. In one embodiment, the method further comprises inducing the mESC-DE cells by said growth promoting factors produces 37% of surfactant protein-C (SP-C) expressing cells. In one embodiment, the first period of time is 6 days. In another embodiment, the second period of time is 9 days.

Another aspect of the present disclosure is directed to a method for generating alveolar type II (AT II) cells, comprising: (a) a two-step (step A and B) differentiation protocol to generate alveolar type II (AT II) cells from one or more embryonic stem cells (mESC), wherein the two-step (step A and B) differential protocol is performed by: (i) A. inducing the embryonic stem cells (mESC) by a definitive endoderm (DE) inducer to produce mESC-DE cells at a first period of time, and (ii) B. inducing the mESC-DE cells by hydrocortisone at a second period of time to generate alveolar type II (AT II) cells.

In one embodiment, the definitive endoderm (DE) inducer is an IDE2 small inducible molecule. In one embodiment, the method further comprises providing embryonic stem cells (mESC) by culturing on one or more gelatin coated dishes.

Another aspect of the present disclosure is directed to a method for generating alveolar type II (AT II) cells, comprising: (a) a two-step (step A and B) differentiation protocol to generate alveolar type II (AT II) cells from one or more embryonic stem cells (mESC), wherein the two-step (step A and B) differential protocol is performed by: (i) A. inducing the embryonic stem cells (mESC) by a definitive endoderm (DE) inducer to produce mESC-DE cells at a first period of time, wherein the definitive endoderm (DE) inducer is an IDE2 inducible molecule, and (ii) B. inducing the mESC-DE cells by a hydrocortisone, a fibroblast growth factor (FGF2), and a conditioned medium of A549 cell line at a second period of time to generate alveolar type II (AT II) cells.

In one embodiment, the method further comprises providing embryonic stem cells (mESC) by culturing on one or more gelatin coated dishes. In one embodiment, the first period is from about 4 to about 8 days. In another embodiment, the second period is from about seven to about eleven days. In a specific embodiment, the first period of time is about 6 days and the second period of time is about 9 days.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF DRAWINGS

FIG. 1 illustrates a method for generating lung epithelial or alveolar type II (AT II) cells from one or more embryonic stem cells (mESCs) according to an embodiment;

FIG. 2 shows a process steps for culturing mESCs in a period of time according to an embodiment;

FIG. 3A and FIG. 3B shows an image of cell showing epithelial morphology characteristic of the alveolar cells assayed by phase contrast microscopy according to an embodiment;

FIG. 4A and FIG. 4B is a graph illustrating an expression levels of markers (Sox 17 and Foxa2) for alveolar cells on real-time polymerase chain reaction (RT-PCR) test;

FIG. 5A and FIG. 5B shows an image of mESC-derived DE cells immunostained by rabbit anti-goat Foxa2 antibody;

FIG. 6A and FIG. 6B shows an image of mESC-derived DE cells immunostained by nuclei counterstained with DAPI;

FIG. 7 is a graph illustrating an expression levels of Foxa2 in mESCs and DE by flow cytometric analysis;

FIG. 8A-8G is a flowchart illustrating a gene expression levels of the markers analyzed at day 0, day 6, and day 15 of differentiation;

FIG. 9A is a graph illustrating a SP-C level in different experiment groups by flow cytometric analyses;

FIG. 9B is a graph illustrating a SP-C level in different experiment groups by immunostaining analyses;

FIG. 10A-10C show images of mESC-derived alveolar type II (AT II) cells by ultrastructural analysis on an electron microscopy;

### DETAILED DESCRIPTION

The present invention generally relates to a method for generating lung epithelial cells, and more particularly relates to a method for generating lung epithelial or alveolar type II (AT II) cells from one or more embryonic stem cells (mESCs).

A description of embodiments of the present invention will now be given with reference to the figures. It is expected that the present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes that come within the meaning and range of equivalency of the claims are to be embraced within their scope.

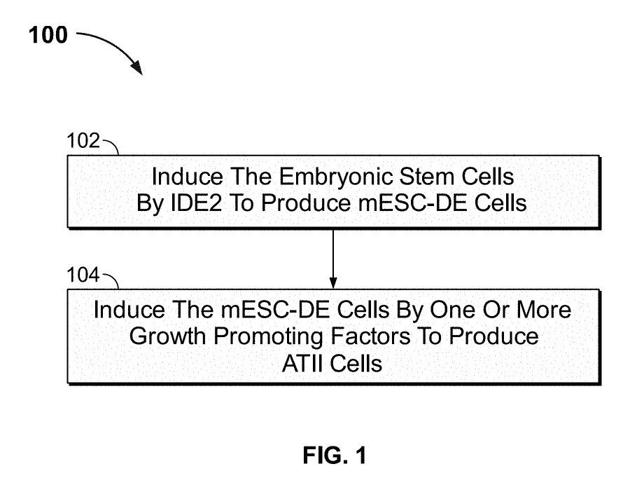

According to an embodiment of the invention, a method 100 for generating alveolar type II (AT II) cells or lung epithelial cells, is shown in FIG. 1. The method 100 comprises a two-step differentiation protocol to generate alveolar type II (AT II) cells from one or more embryonic stem cells (mESC). In some embodiments, the embryonic stem cells (mESC) could be procured from mouse family, or any desired animal. In an embodiment, the two-step differential protocol is performed by step 102 and 104. In one embodiment, the embryonic stem cells (mESC) are induced by a definitive endoderm (DE) inducer to produce mESC-DE cells, at a first period of time in step 102. In step 104, the mESC-DE cells are induced by at least three growth promoting factors at a second period of time to generate alveolar type II (AT II) cells.

In one embodiment, the definitive endoderm (DE) inducer is a small inducible molecule IDE2, in step 102. In step 104, the growth promoting factors are hydrocortisone, fibroblast growth factor (FGF2), and conditioned medium of A549 cell line. In some embodiments, the method further comprises providing embryonic stem cells (mESC) by culturing on one or more gelatin coated dishes. In some embodiments, the first period of time is 6 days, and the second period of time is 9 days. In various embodiments, the method further comprises inducing the mESC-DE cells by said growth promoting factors including hydrocortisone, fibroblast growth factor (FGF2), and conditioned medium of A549 cell line produces 37% of surfactant protein-C (SP-C) expressing cells at step B.

The present disclosure is directed to a method for generating alveolar type II (AT II) cells. The method comprises a two-step differentiation protocol to generate alveolar type II (AT II) cells from one or more embryonic stem cells (mESC). The two-step differential protocol is performed by (a) inducing the embryonic stem cells (mESC) by a definitive endoderm (DE) inducer to produce mESC-DE cells at a first period of time, and (b) inducing the mESC-DE cells by at least three growth promoting factors at a second period of time to generate alveolar type II (AT II) cells.

In another example of this method, the two-step differential protocol is performed by (a) inducing the embryonic stem cells (mESC) by a definitive endoderm (DE) inducer to produce mESC-DE cells at a first period of time, and (b) inducing the mESC-DE cells by hydrocortisone at a second period of time to generate alveolar type II (AT II) cells.

In another embodiment, the method 100 for generating alveolar type II (AT II) cells or lung epithelial cells, is disclosed. The method 100 comprises a two-step differentiation protocol to generate alveolar type II (AT II) cells from one or more embryonic stem cells (mESC). In an embodiment, the two-step differential protocol is performed by step 102 and 104. In one embodiment, the embryonic stem cells (mESC) are induced by a definitive endoderm (DE) inducer to produce mESC-DE cells at a first period of time, in step 102. In step 104, the mESC-DE cells are induced by hydrocortisone alone, at a second period of time to generate alveolar type II (AT II) cells. In some embodiments, the first period of time is 6 days, and the second period of time is 9 days. In some embodiments, the method further comprises providing embryonic stem cells (mESC) by culturing on one or more gelatin coated dishes.

The advantage of the present invention are, a) production of epithelial cells of the lung type II (ATII) from mouse embryonic stem cells (mESCs) via a two-step protocol, b) production of ATII cells from endodermal cells induced by small inducing molecule IDE2, c) production of ATII cells by inductive capacity of hydrocortisone alone from ESCs, without the addition of other growth factors, d) production of AT (II) cells by only four factors: IDE2, Hydrocortisone, FGF2 and A549-CM, e) differentiation of AT (II) cells in shorter duration within 15 days, f) Production of 36.13% of SP-C positive cells, which is a specific marker for the detection of epithelial cells of type II alveolus, and g) a new scientific approach for the treatment of pulmonary disease based on stem cell therapy is achieved.

The method for generating alveolar type II (AT II) cells from the embryonic stem cells (mESC) of a mouse or rabbit, or any animal ensures abundant production of AT (II) cells for the treatment of pulmonary diseases. Further, the main function of AT (II) cells is the production of surfactants, where it could be used for the treatment of acute respiratory distress syndrome (ARDS) and other lungs related diseases. The epithelial cells or alveolar type II (AT II) cells of the lungs possess good potential for drug screening and replacement therapies.

The definitive endoderm (DE) inducer may be an IDE2 small inducible molecule. The growth promoting factors may be hydrocortisone, fibroblast growth factor (FGF2), and conditioned medium of A549 cell line. The method may further comprise providing embryonic stem cells (mESC) by culturing on one or more gelatin coated dishes. The method may further comprise inducing the mESC-DE cells by growth promoting factors produces 37% of surfactant protein-C (SP-C) expressing cells. The first period of time may be for a period of 4-8 days, and in one example for 6 days. The second period of time may be for a period of 7-11 days, and in one example for 9 days. The definitive endoderm (DE) inducer may be an IDE2 small inducible molecule.

The invention is further explained in the following examples, which however, are not to be construed to limit the scope of the invention.

### EXAMPLES

### Example—1 Production of Endoderm (DE)-Like Cells Using Small Molecule IDE2

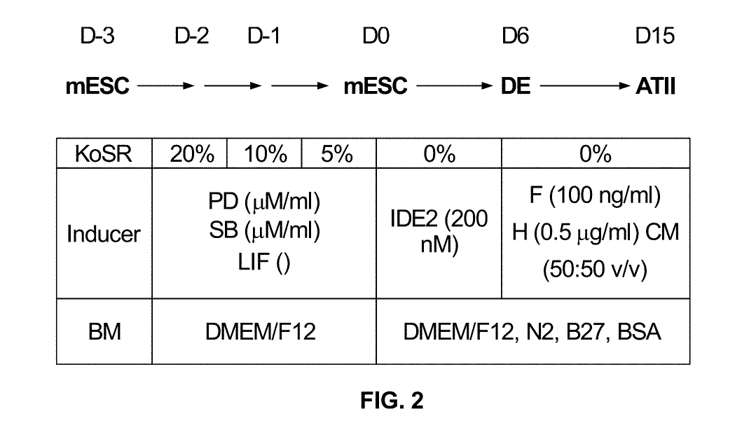

To produce endoderm (DE) like cells, RB20 mESCs (embryonic stem cells) were maintained in adherent culture conditions on gelatin-coated dishes, prior to induction of differentiation. As depicted in FIG. 2, an overview of the mESCs maintenance and differentiation protocol. Differentiation of the cells was initiated via a reduction in concentration of KoSR; after three days, cells were induced by IDE2 for six days. In the next step, DE cells differentiated into alveolar type II-like (AT II-like) cells using seven different combinations of three inductive factors: FGF2 (F), hydrocortisone (H) and A549 conditioned medium (CM) during 9 days. The mESCs were cultured in media with reducing concentration of serum for 3 days, for example, 20%, 10% and 5% fetal bovine serum (FBS) in day one, two and three, respectively. Then, it was induced by 200 nM small inducing molecule IDE2 for 6 days. At the end of this first step, DE like cells were characterized for the expression of two DE markers, Sox17 and Foxa2.

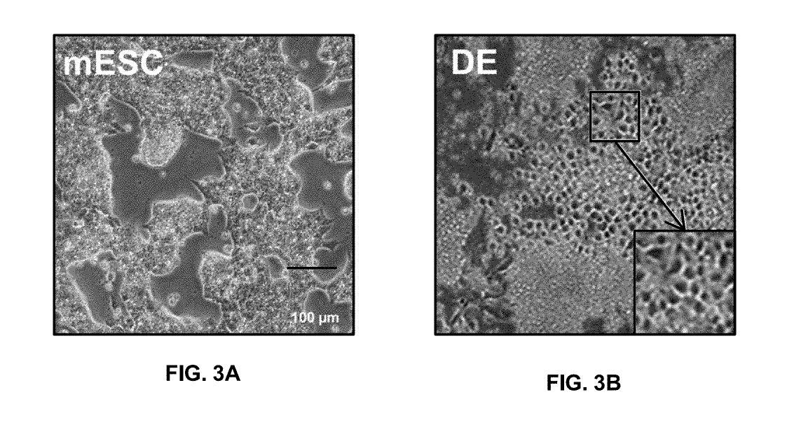

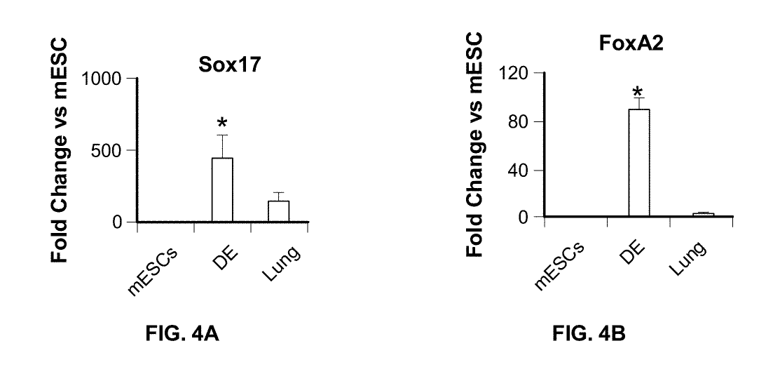

The morphology of the said markers in the DE-like cells was assayed by phase contrast microscopy. At day 6 of IDE2 induction, cells showed the epithelial morphology characteristic of DE cells in phase contrast micrograph, as shown in FIG. 3A and FIG. 3B. Real-time polymerase chain reaction (RT-PCR) results in FIG. 4A and FIG. 4B, showed increased expression levels (*P<0.05) of Sox 17 and Foxa2 at day 6 compared to the mESCs negative control group.

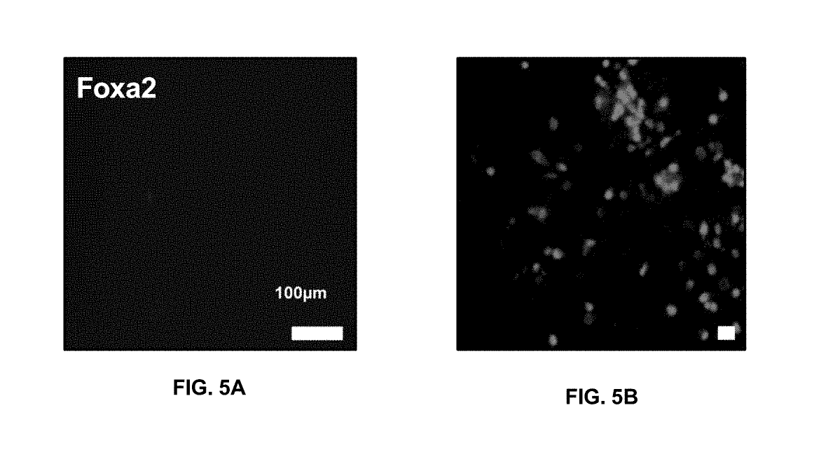

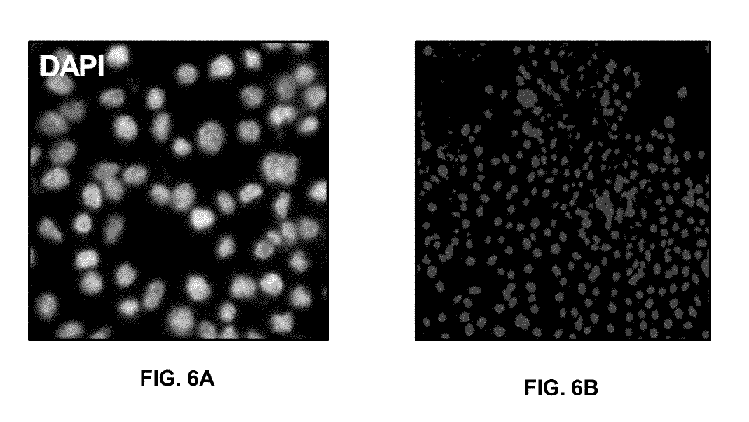

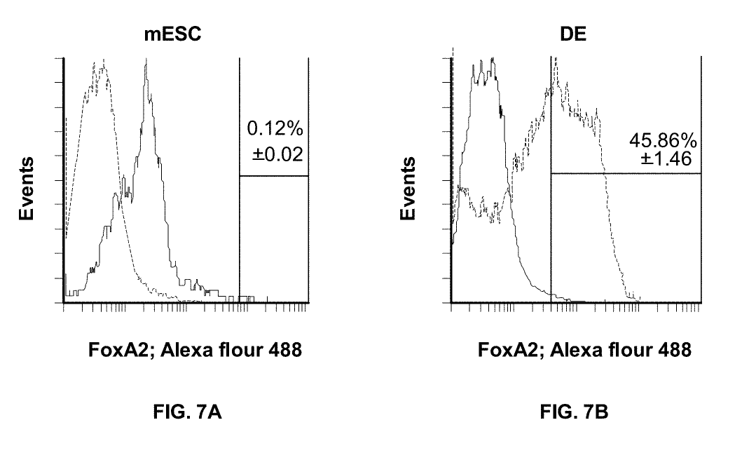

The mESC-derived DE cells were immunostained by rabbit anti-goat Foxa2 antibody, shown in FIG. 5A and FIG. 5B. Lack of expression of Foxa2 could be seen in mESC cells. The mESC-derived DE cells were immunostained by nuclei counterstained with DAPI, shown in FIG. 6A and FIG. 6B. Flow cytometric analysis showed the increase in numbers of cells that expressed DE-specific marker—Foxa2, at the protein level, as shown in FIG. 7.

### Example—2 Induction of mESC-DE Towards Alveolar Type AT (II)-Like Cells Using Hydrocortisone Containing Media

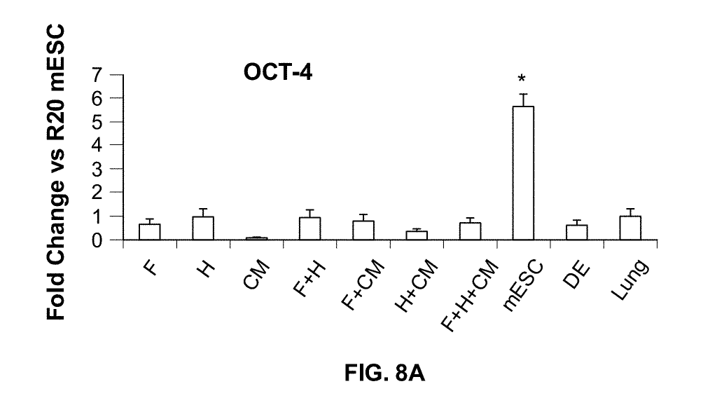

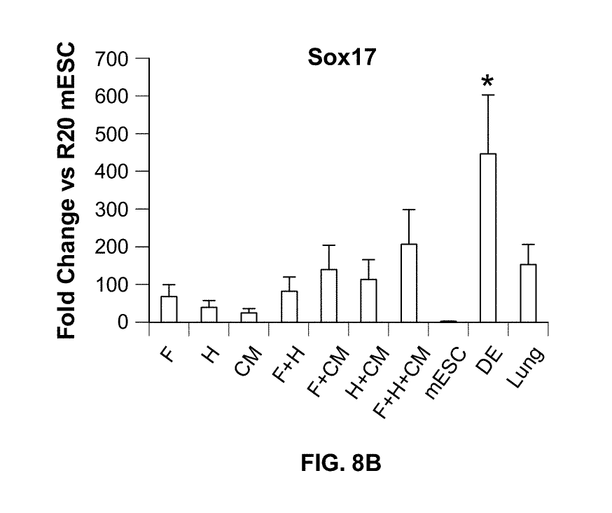

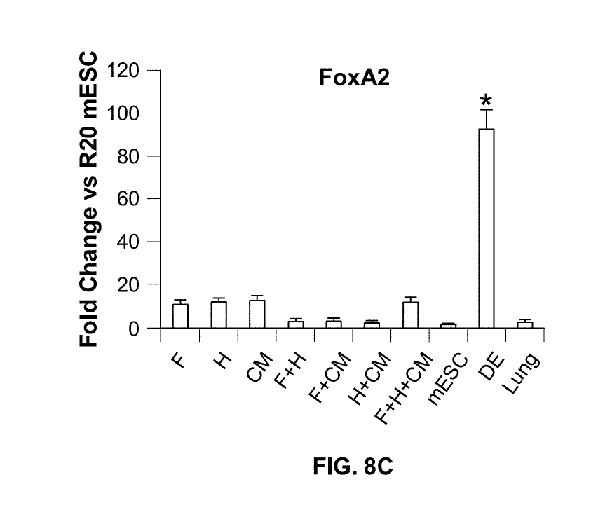

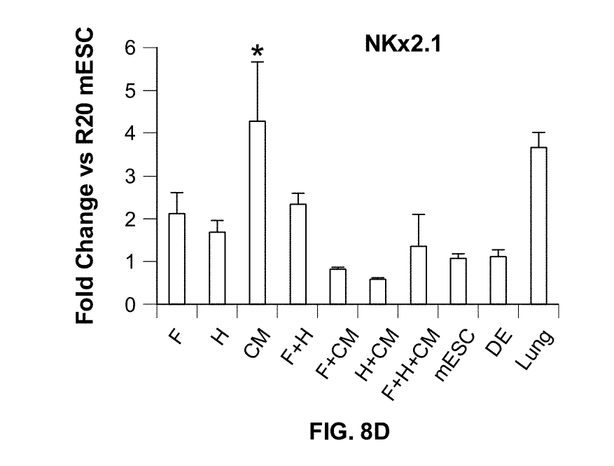

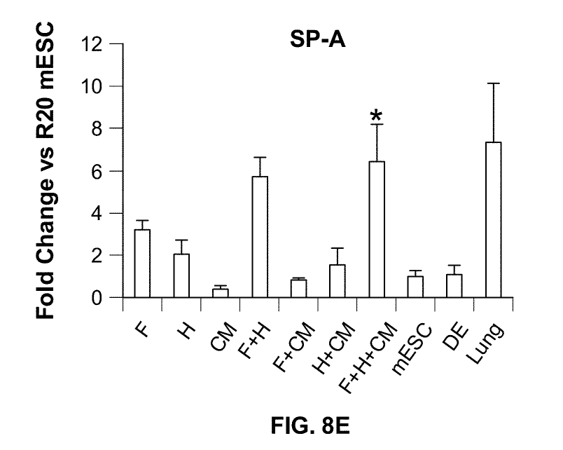

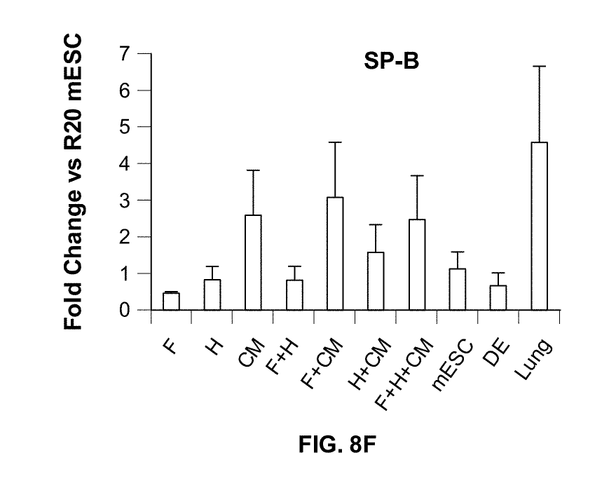

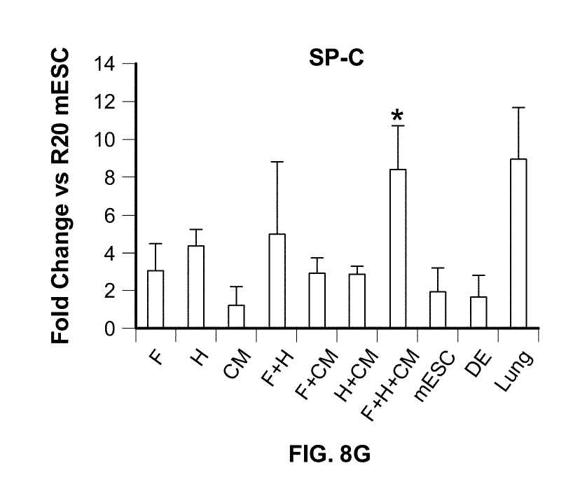

After six days induction with IDE2, DE-like cells were induced with seven different differentiation media as shown in FIG. 2. After 9 days, the resultant cell population for different alveolar type AT (II)-specific markers using gene and protein expression analysis by quantitative RT-PCR, were analyzed as shown in FIG. 8A-8G. Expression levels of pluripotency (A), DE (B and C) and lung alveolar (D-G) specific marker genes were analyzed during different stages of differentiation (mESCs, DE and ATII) and in different experimental groups. The target gene expression level was normalized to GAPDH and presented relative to mESCs. Data are presented as mean±SD.

Significant to mESCs and DE groups, but not significant with positive control (lung) group. At least P<0.05 as determined by ANOVA with Turkey's HSD test, n=3, F: FGF2, H: Hydrocortisone, CM: A594 conditioned medium, mESC: Mouse embryonic stem cells as the negative control: Definitive endoderm-like cells, ATII: Lung alveolar type II-like cells. In all cases, the results were compared to DE-like cells at day 6, and mESCs at day 0. The morphology of the resultant cells were investigated by the phase contrast microscopy, and ultrastructural analysis by electron microscopy.

A. Gene Expression Profile of Differentiated ATII-Like Cells:

The gene expression levels of pluripotent marker Oct4, DE-specific markers Sox17 and Foxa2 and several important early and late ATII-specific genes (Nkx2.1, SP-A, SP-B, SP-C and SP-D) were analyzed at days 0, 6, and 15 of differentiation, as shown in FIG. 8A-8G. For gene expression experiments, undifferentiated ESCs were used as the negative control and lung tissue as the positive control.

The results showed downregulation of Oct4, as a pluripotent marker, in all experimental groups compared with ESCs, as shown in FIG. 8A. DE-specific markers such as Sox17 and Foxa2 significantly upregulated in the DE stage, and subsequently downregulated after further induction towards ATII cells with different media, as shown in FIG. 8B and FIG. 8C. Gene expression analysis at day 15, ATII-like cells showed significant upregulation of ATII-specific markers such as SP-A and SP-C in the FGF2, hydrocortisone and conditioned medium of A549 (F+H+CM) group. Nkx2.2, the primary marker of alveolar differentiation, upregulated in CM, as shown in FIG. 8D-8G.

B. SP-C Protein Expression Level in Differentiated AT (II)-Like Cells:

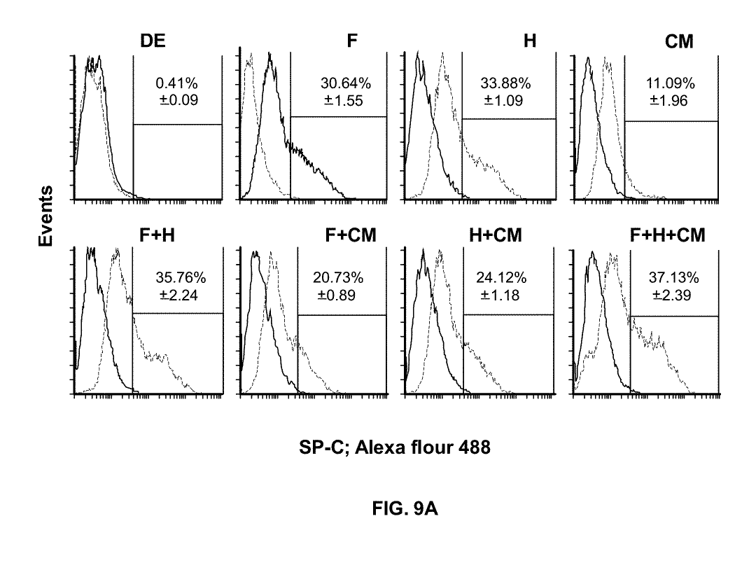

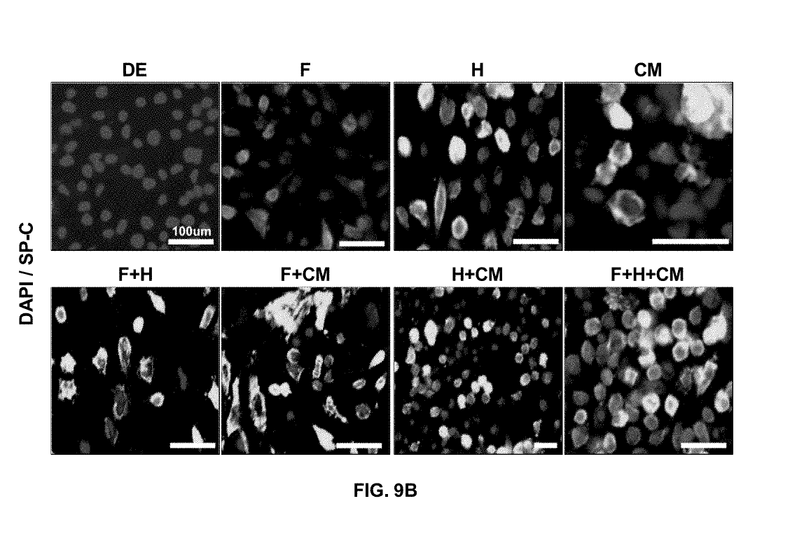

SP-C, a unique marker of AT (II) cells, has been commonly used to identify these cells from other lung parenchymal cell types. Flow cytometric and immunostaining analyses were performed to determine the level of SP-C in different experimental groups, as shown in FIG. 9A and FIG. 9B. In FIG. 9A, the numbers of SP-C positive cells were investigated in different stages of differentiation (mESCs, DE and ATII) and different experimental groups.

All F and H groups showed increased numbers of SP-C positive cells. The highest positive number of SP-C cells belonged to the F+H+CM group. Data are presented as mean±SD. In FIG. 9B, Cells in different stages of differentiation (mESCs, DE and ATII) and different experimental groups, immunostained by rabbit anti-goat SP-C antibody and counterstained with DAPI. The results were consistent with the results of flow cytometric analysis. F: FGF2, H: Hydrocortisone, CM: A594 conditioned medium, mESC: Mouse embryonic stem cells as negative control, DE: Definitive endoderm-like cells, ATII: Lung alveolar type II-like cells. While the number of SP-C positive cells was hardly detectable in day 0 mESCs (0.44±0.07%), and at day 6 DE-like cells (0.41±0.09%), in other differentiation protocols, SP-C positive cells were detected. As determined by flow cytometry, the number of SP-C positive cells was highest (37.13±2.39%) in F+H+CM compared to the other groups FIG. 9A.

C. Ultra-Morphology of mESC-Derived AT (II)-Like Cells:

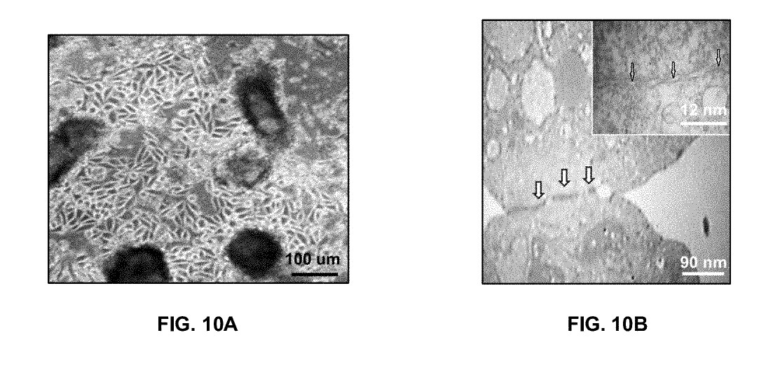

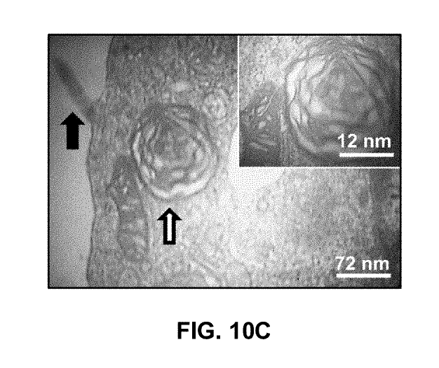

Mouse ESCs induced for 15 days in FGF2, hydrocortisone and A549 conditioned medium (F+H+CM) were analyzed by phase contrast microscopy and transmission electron microscopy (TEM). The Morphology of AT (II)-like cells at day 15 of culture in the F+H+CM group, is shown in FIG. 10A. The epithelial morphology of day 15 AT (II)-like cells showed lateral cell-cell contacts, which included tight junctions, and higher magnification also showed these structures by transmission electron microscopy (TEM), as shown in FIG. 10B and FIG. 10C. Ultrastructure of day 15 ATII-like cell which shows microvilli and lamellar body. Higher magnification showed a well-developed lamellar body with electron dense lamellae in a multi-vesicular body, is shown in FIG. 10C.

Another aspect of the present disclosure is directed to a method for generating alveolar type II (AT II) cells, comprising a two-step (step A and B) differentiation protocol to generate alveolar type II (AT II) cells from one or more embryonic stem cells (mESC). The two-step (step A and B) differential protocol is performed by: (A) inducing the embryonic stem cells (mESC) by a definitive endoderm (DE) inducer to produce mESC-DE cells at a first period of time, wherein the definitive endoderm (DE) inducer is an IDE2 inducible molecule, and (B) inducing the mESC-DE cells by a hydrocortisone, a fibroblast growth factor (FGF2), and a conditioned medium of A549 cell line at a second period of time to generate alveolar type II (AT II) cells. The method may further comprise providing embryonic stem cells (mESC) by culturing on one or more gelatin coated dishes. In one example, the first period is from about 4 to about 8 days. In another example, the second period is from about seven to about eleven days. The first period of time may be about 6 days and the second period of time may be about 9 days.

Table 1 shows the real time RT-PCR primers. Table 2 shows the primary and secondary antibodies used for immunofluorescent staining and flow cytometry analyses.

TABLE 1
Real-Time RT-PCR Primers

| | Forward Primer | Reverse Primer | |
|---|---|---|---|
| | (Sequences | (Sequences | Length |
| Genes | 5′→3′) | 5′→3′) | (bp) |
| Sftpa | GCAAACAATGGGAGT | CGGCTCTGGTACACA | 119 |
| (SP-A) | CCTC | TCTCTC | |
| | (SEQ ID NO: 1) | (SEQ ID NO: 2) | |
| Sftpb | GCTAGACAGGCAAAA | GGTGCAGGCTGAGGC | 210 |
| (SP-B) | GTGTGAAC | TTGTC | |
| | (SEQ ID NO: 3) | (SEQ ID NO: 4) | |
| Sftpc | ACCCTGTGTGGAGAG | TTTGCGGAGGGTCTT | 88 |
| (SP-C) | CTACCA | TCCT | |
| | (SEQ ID NO: 5) | (SEQ ID NO: 6) | |
| Sftpd | AGACAGAGGAATCAA | AGGGAACAATGCAGC | 133 |
| (SP-D) | AGGTG | TTTCTGA | |
| | (SEQ ID NO: 7) | (SEQ ID NO: 8) | |
| Nkx2.1 | TTCCCCGCCATCTCC | TGTTCTTGCTCACGT | 94 |
| | CGCT | CCCCCA | |
| | (SEQ ID NO: 9) | (SEQ ID NO: 10) | |
| FoxA2 | CGAGTTAAAGTATGC | CTATGTGTTCATGCC | 123 |
| | TGGGAG | CATTCATC | |
| | (SEQ ID NO: 11) | (SEQ ID NO: 12) | |
| Sox17 | GATGTAAAGGTGAAA | AAGACTTGCCTAGCA | 196 |
| | GGCGA | TCTTG | |
| | (SEQ ID NO: 13) | (SEQ ID NO: 14) | |
| Oct-4 | GAACTAGCATTGAGA | CATACTCGAACCACA | 129 |
| | ACCGT | TCCTTC | |
| | (SEQ ID NO: 15) | (SEQ ID NO: 16) | |
| Gapdh | CAACTCCCACTCTTC | GCAGCGAACTTTATT | 125 |
| | CACTT | GATGGGA | |
| | (SEQ ID NO: 17) | (SEQ ID NO: 18) | |

TABLE 2
Primary and Secondary Antibodies used for Immunofluorescent
Staining and Flow Cytometry Analyses

| | | | | |
|---|---|---|---|---|
| | Host | | Catalog | Dilution |
| Antibody name | species | Manufacturer | number | factor |

Primary antibodies

| | | | | |
|---|---|---|---|---|
| Oct-4 | Rabbit | Sigma Aldrich | AB3209 | 1:100 |
| Foxa2 | Rabbit | Sigma Aldrich | AB4125 | 1:100 |
| Surfactant protein-C | Rabbit | Chemicon | ABC99 | 1:500 |
| (SP-C) | | | | |

Secondary antibodies

| | | | | |
|---|---|---|---|---|
| goat IgG-alexa fluor | Rabbit | Invitrogen | A-11008 | 1:1000 |
| 488 | | | | |
| goat IgG-alexa fluor | Rabbit | Invitrogen | R37117 | 1:1000 |
| 594 | | | | |

The foregoing description comprise illustrative embodiments of the present invention. Having thus described exemplary embodiments of the present invention, it should be noted by those skilled in the art that the within disclosures are exemplary only, and that various other alternatives, adaptations, and modifications may be made within the scope of the present invention. Merely listing or numbering the steps of a method in a certain order does not constitute any limitation on the order of the steps of that method.

Many modifications and other embodiments of the invention will come to mind to one skilled in the art to which this invention pertains having the benefit of the teachings presented in the foregoing descriptions. Although specific terms may be employed herein, they are used only in generic and descriptive sense and not for purposes of limitation. Accordingly, the present invention is not limited to the specific embodiments illustrated herein.

While the above is a complete description of the preferred embodiments of the invention, various alternatives, modifications, and equivalents may be used. Therefore, the above description and the examples should not be taken as limiting the scope of the invention, which is defined by the appended claims.

1. A method for generating alveolar type II (AT II) cells, comprising:
a two-step (step A and B) differentiation protocol to generate alveolar type II (AT II) cells from one or more embryonic stem cells (mESC), wherein the two-step (step A and B) differential protocol is performed by:
A. inducing the embryonic stem cells (mESC) by a definitive endoderm (DE) inducer to produce mESC-DE cells at a first period of time, and
B. inducing the mESC-DE cells by at least three growth promoting factors at a second period of time to generate alveolar type II (AT II) cells.

2. The method of claim 1, wherein the definitive endoderm (DE) inducer is an IDE2 small inducible molecule.

3. The method of claim 1, wherein the growth promoting factors are hydrocortisone, fibroblast growth factor (FGF2), and conditioned medium of A549 cell line.

4. The method of claim 1, further comprises providing embryonic stem cells (mESC) by culturing on one or more gelatin coated dishes.

5. The method of claim 1, further comprises inducing the mESC-DE cells by said growth promoting factors produces 37% of surfactant protein-C (SP-C) expressing cells.

6. The method of claim 1, wherein the first period of time is 6 days.

7. The method of claim 1, wherein the second period of time is 9 days.

8. A method for generating alveolar type II (AT II) cells, comprising:
a two-step (step A and B) differentiation protocol to generate alveolar type II (AT II) cells from one or more embryonic stem cells (mESC), wherein the two-step (step A and B) differential protocol is performed by:
A. inducing the embryonic stem cells (mESC) by a definitive endoderm (DE) inducer to produce mESC-DE cells at a first period of time, and
B. inducing the mESC-DE cells by hydrocortisone at a second period of time to generate alveolar type II (AT II) cells.

9. The method of claim 8, wherein the definitive endoderm (DE) inducer is an IDE2 small inducible molecule.

10. The method of claim 8, further comprises providing embryonic stem cells (mESC) by culturing on one or more gelatin coated dishes.

11. The method of claim 8, wherein the first period of time is 6 days.

12. The method of claim 8, wherein the second period of time is 9 days.

13. A method for generating alveolar type II (AT II) cells, comprising:
a two-step (step A and B) differentiation protocol to generate alveolar type II (AT II) cells from one or more embryonic stem cells (mESC), wherein the two-step (step A and B) differential protocol is performed by:
A. inducing the embryonic stem cells (mESC) by a definitive endoderm (DE) inducer to produce mESC-DE cells at a first period of time, wherein the definitive endoderm (DE) inducer is an IDE2 inducible molecule, and
B. inducing the mESC-DE cells by a hydrocortisone, a fibroblast growth factor (FGF2), and a conditioned medium of A549 cell line at a second period of time to generate alveolar type II (AT II) cells.

14. The method of claim 13, further comprises providing embryonic stem cells (mESC) by culturing on one or more gelatin coated dishes.

15. The method of claim 13, wherein the first period of time is 6 days.

16. The method of claim 13, wherein the second period of time is 9 days.


SEQUENCE LISTING (18 sequences)

SEQ ID NO: 1 - DNA, 19 bp, Mouse
gcaaacaatgggagtcctc

SEQ ID NO: 2 - DNA, 21 bp, Mouse
cggctctggtacacatctctc

SEQ ID NO: 3 - DNA, 23 bp, Mouse
gctagacaggcaaaagtgtgaac

SEQ ID NO: 4 - DNA, 20 bp, Mouse
ggtgcaggctgaggcttgtc

SEQ ID NO: 5 - DNA, 21 bp, Mouse
accctgtgtggagagctacca

SEQ ID NO: 6 - DNA, 19 bp, Mouse
tttgcggagggtctttcct

SEQ ID NO: 7 - DNA, 20 bp, Mouse
agacagaggaatcaaaggtg

SEQ ID NO: 8 - DNA, 22 bp, Mouse
agggaacaatgcagctttctga

SEQ ID NO: 9 - DNA, 19 bp, Mouse
ttccccgccatctcccgct

SEQ ID NO: 10 - DNA, 21 bp, Mouse
tgttcttgctcacgtccccca

SEQ ID NO: 11 - DNA, 21 bp, Mouse
cgagttaaagtatgctgggag

SEQ ID NO: 12 - DNA, 23 bp, Mouse
ctatgtgttcatgcccattcatc

SEQ ID NO: 13 - DNA, 20 bp, Mouse
gatgtaaaggtgaaaggcga

SEQ ID NO: 14 - DNA, 20 bp, Mouse
aagacttgcctagcatcttg

SEQ ID NO: 15 - DNA, 20 bp, Mouse
gaactagcattgagaaccgt

SEQ ID NO: 16 - DNA, 21 bp, Mouse
catactcgaaccacatccttc

SEQ ID NO: 17 - DNA, 20 bp, Mouse
caactcccactcttccactt

SEQ ID NO: 18 - DNA, 22 bp, Mouse
gcagcgaactttattgatggga